(19) United States
(12) Patent Application Publication
(10) Pub. No.: US 2022/0008015 A1
(43) Pub. Date: Jan. 13, 2022

(54) MAT FOR DETECTING BIOLOGICAL INFORMATION, DEVICE FOR DETECTING BIOLOGICAL INFORMATION, AND SYSTEM FOR DISTRIBUTING BIOLOGICAL INFORMATION

(71) Applicant: LIQUID DESIGN SYSTEMS INC., Yokohama-shi, Kanagawa (JP)

(72) Inventors: Naoya TOHYAMA, Yokohama-shi, Kanagawa (JP); Yasuchika AOKI, Yokohama-shi, Kanagawa (JP)

(21) Appl. No.: 17/255,276

(22) PCT Filed: Jun. 24, 2019
(86) PCT No.: PCT/JP2019/025020

§ 371 Date: Sep. 16, 2021

(30) Foreign Application Priority Data
Jun. 22, 2018 (JP) 2018-119301

(51) Int. Cl. A61B 5/00; A61B 5/08; A61B 5/113

(52) U.S. Cl. CPC A61B 5/6892; A61B 2503/04; A61B 5/113; A61B 5/0816

(57) ABSTRACT

A mat for detecting biological information has an air bag formed in an elongated shape and outputting air according to pressure from a living body, a plate-shaped cushion member in which a concave portion having substantially the same size as the air bag is formed in a central portion, the air bag is fitted in the concave portion, and has substantially the same thickness as the air bag, and a pair of synthetic resin plate-shaped members each having substantially the same size as the plate-shaped cushion member and being formed so as to be bendable as a whole and provided so as to sandwich the plate-shaped cushion member.

### TECHNICAL FIELD

The present invention relates to a mat for detecting biological information, a device for detecting biological information, and a system for distributing biological information.

### BACKGROUND ART

Conventionally, a biological information collection device for collecting biological information of a human body without attaching an electrode, a lead wire, or the like to a human body has been disclosed (see PTL1). The technique of PTL1 can measure breathing, heart rate, and the like by detecting air pressure in an air bladder by an omnidirectional microphone or a pressure sensor even when a human body is placed at any position on a plate-shaped member.

### CITATION LIST

### Patent Literature

PTL1: Japanese Patent No. 3,419,732

### SUMMARY OF THE INVENTION

### Technical Problems

In the technique of PTL1, a plate-shaped member having a sufficient width to allow a human body to ride is required. However, it is not easy to prepare a plate-shaped member having a size that allows a human body to ride, and it is inconvenient to carry it.

On the other hand, if the size of the plate-shaped member is reduced, there is a problem that biological information of the human body cannot be accurately detected when a mattress or other carpet is interposed between the plate-shaped member and the human body.

In view of such circumstances, it is an object of the present invention to provide a mat for detecting biological information, a device for detecting biological information, and a system for distributing biological information capable of detecting periodic biological information of a subject with high accuracy.

### Solution to the Problems

A mat for detecting biological information has an air bag formed in an elongated shape and outputting air according to pressure from a living body, a plate-shaped cushion member in which a concave portion having substantially the same size as the air bag is formed in a central portion, the air bag is fitted in the concave portion, and has substantially the same thickness as the air bag, and a pair of synthetic resin plate-shaped members each having substantially the same size as the plate-shaped cushion member and being formed so as to be bendable as a whole and provided so as to sandwich the plate-shaped cushion member.

A device for detecting biological information according to the present invention has the mat for detecting biological information disposed below a subject and a biological information detection unit for detecting periodic biological information of a subject based on air pressure from the mat for detecting biological information.

### Effect of the Invention

The present invention can detect periodic biological information of a subject with high accuracy.

### BRIEF DESCRIPTION OF DRAWINGS

FIG. 1 is a front view showing a schematic configuration of a biological information detection apparatus according to the first embodiment.

FIG. 2 is a diagram illustrating a configuration of a sensor mat of a biological information detection apparatus.

FIG. 3 is an exploded perspective view showing the configuration of the sensor board.

FIG. 4 is a block diagram showing a functional configuration of the main body.

FIG. 5 is a waveform diagram of a breathing signal when a single air pad is disposed under a carpet in the case where the weight of the measurement object is 2 kg.

FIG. 6 is a waveform diagram of a breathing signal when a sensor mat is disposed under a carpet in the case where the weight of the measurement object is 2 kg.

FIG. 7 is a waveform diagram of a breathing signal when a single air pad is disposed under a carpet when the weight of the measurement object is 6 kg.

FIG. 8 is a waveform diagram of a breathing signal when a sensor mat is disposed under a carpet in the case where the weight of the measurement object is 6 kg.

FIG. 9 is a waveform diagram of the heart rate signal when the air pad alone is placed under the rug.

FIG. 10 is a waveform diagram of a heartbeat signal in the case where a sensor mat is disposed under a carpet.

FIG. 11 is a diagram showing a configuration of a biological information communication system according to the second embodiment.

FIG. 12 is a front view showing the sensor mat cover.

FIG. 13 is a perspective view showing the sensor mat cover.

### DESCRIPTION OF EMBODYMENTS

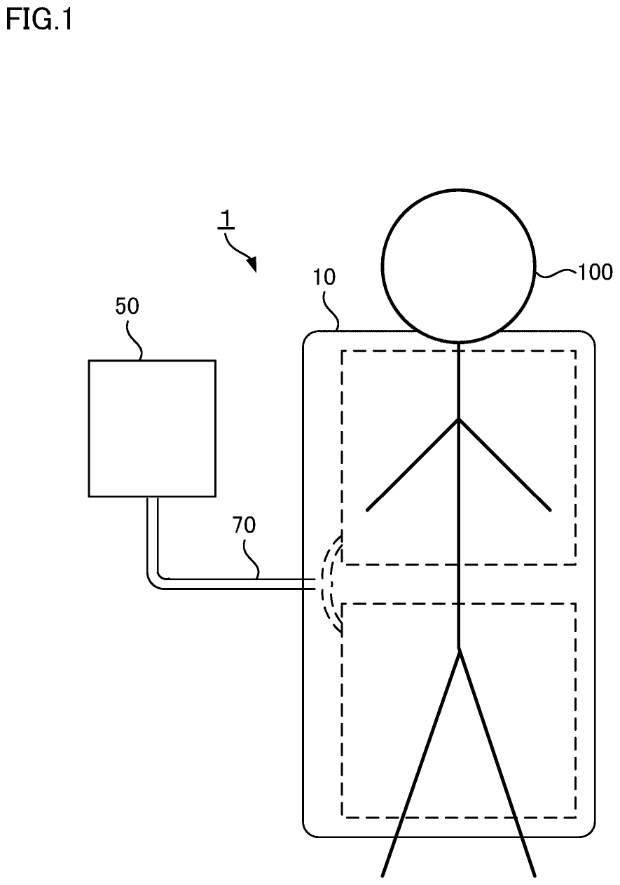

Hereinafter, embodiments of the present invention will be described in detail with reference to the drawings.

[A first embodiment] FIG. 1 is a front view showing the schematic composition of the biological information detection device 1 concerning a first embodiment. The biological information detecting device 1 detects biological information such as a heart rate and a respiration rate from a detection target person of biological information (hereinafter referred to as a “target person”) and performs data communication with an external device. In the present embodiment, the biological information detecting device 1 detects, for example, the breathing rate per minute of the subject 100, such as an infant or an infant, during sleep.

The biological information detecting device 1 includes a sensor mat 10 disposed under a sleeping subject 100, a main body 50 that calculates the respiration rate of the subject 100 based on the air pressure output from the sensor mat 10, and a silicon tube 70 that supplies the air output from the sensor mat 10 to the main body 50. Although not shown, a mattress or the like is placed between the subject 100 and the sensor mat 10.

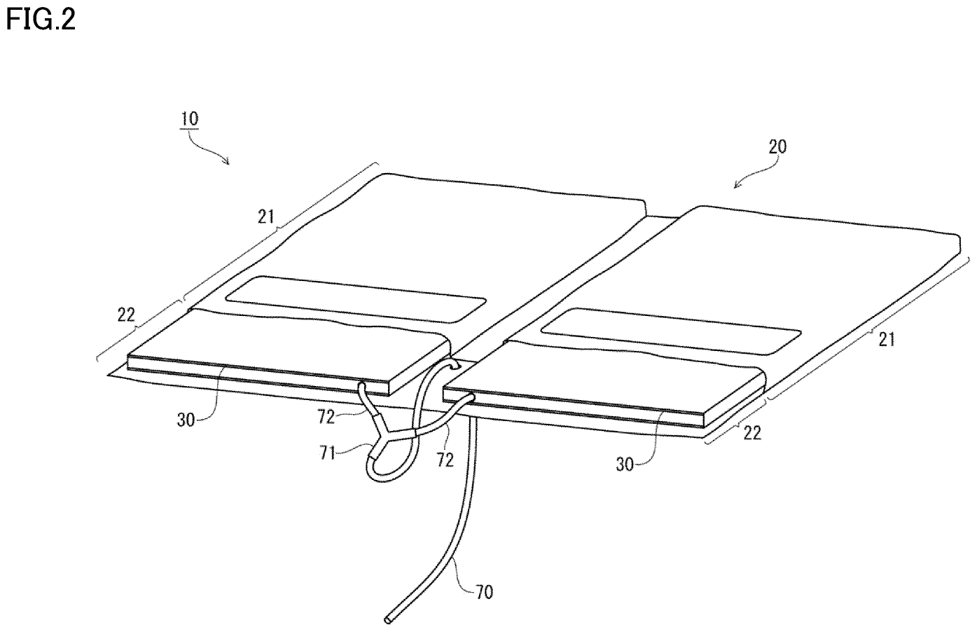

FIG. 2 is a diagram showing a configuration of the sensor mat 10.

The sensor mat 10 includes a sensor mat cover 20 and two sensor boards 30 which are housed in the sensor mat cover 20. Incidentally, the end portion of the silicon tube 70 (sensor mat 10 side), Y-shaped connector 71 is joined is a Y-shaped tube.

On the other hand, the air ports of each sensor board 30 are joined to the junction of the remaining two locations of the Y-shaped connector 71, respectively, via a silicon tube 72. Therefore, the air discharged from each sensor board 30 merges with a Y-shaped connector through each silicon tube 72, is supplied to the main body portion 50 shown in FIG. 1 through the silicon tube 70.

Sensor mat cover 20 is a cover configured in a rectangular shape, two bag portions 21 in which the two sensor boards 30 are respectively housed, and a lid portion 22 which functions so that the sensor board 30 is not outside the bag portion 21.

Each bag portion 21 is configured to have a shape and size enough to fully accommodate one sensor board 30. Then, the bag portions 21 are arranged so as to be symmetrical with respect to the central axis perpendicular to the central portion in the longitudinal direction of the sensor mat cover 20. Therefore, the sensor mat cover 20 can be folded into two by the above-mentioned central axis in a state in which the sensor board 30 is accommodated in each bag portion 21.

The sensor board 30 is placed under the carpet with the subject 100. The sensor board 30 outputs air corresponding to the movement of the subject 100, such as breathing movement or pulsation, and supplies the air to the main body 50 via the silicon tube 70 or the like.

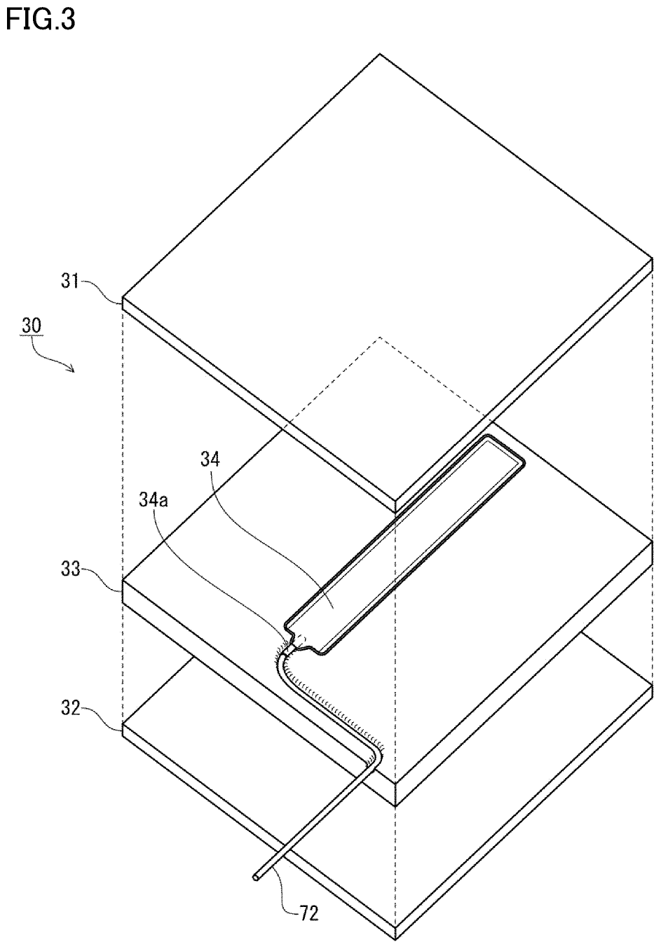

FIG. 3 is an exploded perspective view showing the configuration of the sensor board 30. The sensor board 30 is a plate-shaped member having a substantially square shape, and outputs air in accordance with a movement (pressure) such as breathing movement or pulsation of the subject 100. The sensor board 30 includes a first polyvinyl chloride plate 31, a second polyvinyl chloride plate 32, a cushion member 33, and an air pad 34.

The first and second polyvinyl chloride plates 31 and 32 are, for example, polyvinyl chloride formed in a substantially square plate shape. The first and second polyvinyl chloride plates 31 and 32 have hard surfaces and are formed to be bendable as a whole. The thickness of the first and second polyvinyl chloride plates 31 and 32 is, for example, 1 mm. The corners of the first and second polyvinyl chloride plates 31 and 32 are arched, for example, 5 mm.

It is also conceivable to use an acrylic plate or a veneer plate instead of the first and second polyvinyl chloride plates 31 and 32 However, since the veneer plate is not bendable, it is not suitable for substituting the first and second polyvinyl chloride plates 31 and 32. On the other hand, the acrylic plate can be substituted for the first and second polyvinyl chloride plates 31 and 32 as long as the acrylic plate is formed to have a bendable thickness. In other words, as a substitute for the first and second polyvinyl chloride plates 31 and 32, it may be a plate-shaped member of synthetic resin material which is formed so as to be curved as a whole. Examples of such a synthetic resin material include polypropylene (PP), polyethylene terephthalate (PET), and the like in addition to those described above.

The cushion member 33 is formed of a polyurethane material having a thickness of, for example, 5 mm. The cushion member 33 is formed in a square plate shape having substantially the same size as the first and second polyvinyl chloride plates 31 and 32.

The central portion along the axis of symmetry of the cushion member 33, the recess 33a of the size so that the air pad 34 becomes jammed is formed. When the air pad 34 is fitted into the recess 33a of the cushion member 33, the surface of the air pad 34 becomes substantially the same position as the surface of the cushion member 33.

Air pad 34 is an air bag formed in an elongated shape. The air port of the air pad 34, the air pipe 34a of a predetermined length is provided. One side of the air pipe 34a is inside the air pad 34. The other side of the air pipe 34a is exposed to the outside of the air pad 34 and bonded to the silicon tube 72.

In addition, a cushion member made of a polyurethane material is contained inside the air pad 34 Therefore, the air pad 34 has a predetermined thickness (10 mm in the present embodiment) even in a normal state (a state in which there is no external pressure), and has air inside. When external pressure from the subject 100 is applied to the air pad 34, air is output from the air pad 34 in response to the external pressure. The air output from the air pad 34 is supplied to the main body 50 via the silicon tube 70.

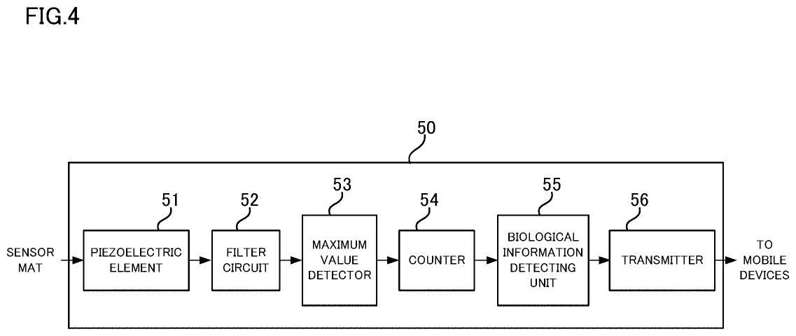

FIG. 4 is a block diagram showing a functional configuration of the main body 50.

The main body 50 includes a piezoelectric element 51, a filter circuit 52, a maximum value detector 53, a counter 54, a biological information detector 55, and a transmitter 56.

Piezoelectric element 51 outputs a signal corresponding to the vibration of the air supplied from the silicon tube 70. Filter circuit 52 removes a predetermined band component of the signal supplied from the piezoelectric element 51. The filter characteristic of the filter circuit 52 is set to a characteristic suitable for extracting the breathing component of the human body from the signal output from the piezoelectric element 51. Therefore, the filter circuit 52 removes the noise component and outputs a breathing signal. It should be noted that the frequency characteristic of the filter circuit 52 can be changed in accordance with the biological information to be detected. For example, when the filter circuit 52 is a high-pass filter (e.g., to remove band components of 3 Hz or less), a signal of other biological information (e.g., a heartbeat signal) is obtained instead of the breathing signal.

The maximum value detector 53 outputs a positive pulse every time the maximum value (peak value) of the breathing signal supplied from the filter circuit 52 is detected. Counter 54 has a timer function and counts the positive polarity pulse supplied from the maximum value detector 53, and resets every predetermined time (e.g., 10 seconds). Then, the counter 54 supplies the count value at that point to the biological information detecting unit 55 every time the reset.

The biological information detecting unit 55 calculates a value obtained by multiplying the number of breaths supplied from the counter 54 by six, and supplies the calculated value to the transmitter 56 as the number of breaths per minute. The transmitter 56 transmits the respiration rate supplied from the biological information detecting unit 55 to the information terminal apparatus of the operator. Thus, the operator can confirm the breathing rate of the subject 100 in real time.

The information terminal apparatus may issue an alarm when the respiration rate of the subject 100 continuously falls below a threshold value for a predetermined time or longer. As a result, the operator can immediately grasp the state in which the respiration rate of the subject 100 is not detected.

As described above, the biological information detection device 1 according to the present embodiment detects the respiration rate in real time for the subject 100 on the sensor mat 10, and transmitting the respiration rate to the information terminal device of the operator. As a result, the operator can grasp the breathing rate of the subject 100 in real time.

Further, even when the subject 100 is on a rug such as a mattress, the biological information detection device 1 can accurately detect the breathing rate of the subject 100 by arranging the sensor mat 10 shown in FIG. 3 below the rug.

[Comparative measurement 1] We measured the respiration signal (output signal of filter circuit 52) when the air pad alone was placed under the rug, and the respiration signal when the sensor mat 10 shown in FIG. 3 was placed under the rug. The measurement conditions were as follows.

The air pad alone and the sensor mat 10 are individually measured.

The air pad alone is the same as the air pad 34 in the sensor mat 10.

The first and second polyvinyl chloride plates 31 and 32 of the sensor mat 10 have a size of 40 cm×40 cm and a thickness of 1 mm.

The thickness of the cushion member 33 (polyurethane material) of the sensor mat 10 is set to 5 mm.

A 2.5 cm thick covering is placed on each of the air pad alone and the sensor mat 10.

A respiratory simulator is placed 20 cm from the center of the air pad on the carpet.

The average respiratory rate of the respiratory simulator is 30 [cycles/minute].

Two types of weight to be measured are used: 2 kg and 6 kg.

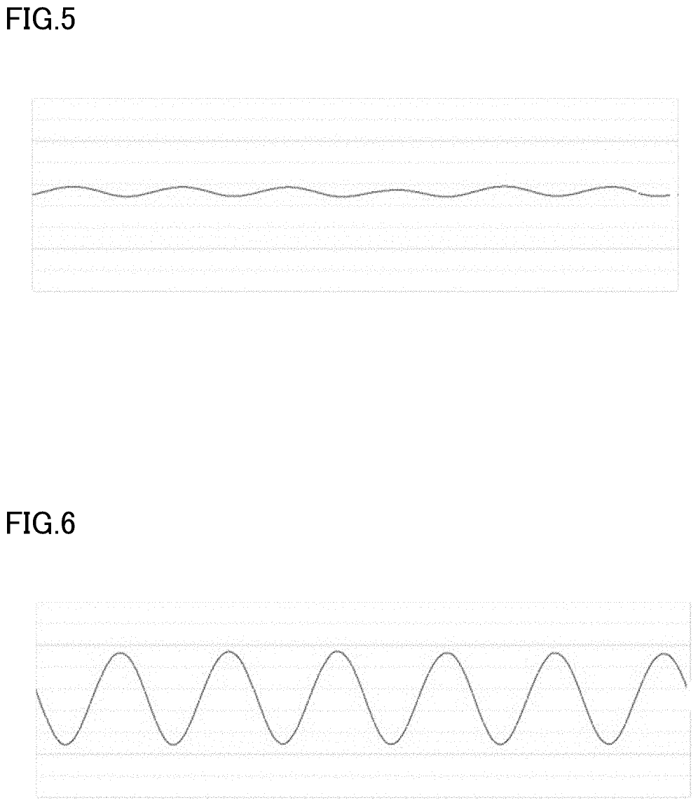

FIG. 5 is a waveform diagram of a breathing signal (an output signal of the filter circuit 52) when an air pad alone is disposed under a carpet in the case where the weight of the object to be measured is 2 kg. FIG. 6 is a waveform diagram of a breathing signal when the sensor mat 10 shown in FIG. 3 is disposed under a carpet in the case where the weight of the measurement object is 2 kg.

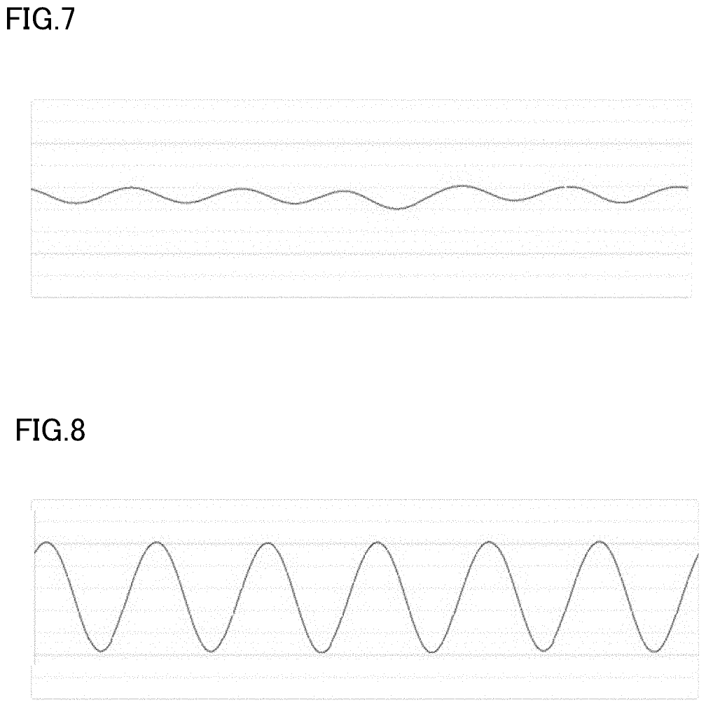

FIG. 7 is a waveform diagram of a breathing signal when a single air pad is disposed under a carpet when the weight of the measurement object is 6 kg. FIG. 8 is a waveform diagram of a respiration signal when the sensor mat 10 shown in FIG. 3 is disposed under a carpet in the case where the weight of the measurement object is 6 kg. In FIGS. 5-8, the vertical axis direction represents the magnitude of the signal, the horizontal axis direction represents time.

According to FIGS. 5 to 8, in the case of both 2 kg and 6 kg of the weight to be measured, the breathing signal obtained from the sensor mat 10 has a larger amplitude than the breathing signal obtained from the air pad alone. That is, the respiration detection sensitivity of the sensor mat 10 was higher than that of the air pad alone.

As described above, the biological information detection apparatus 1 according to the embodiment of the present invention can detect the respiration rate of the subject 100 without using a plate-shaped member having a size capable of covering the entire length of the subject 100 by using the sensor mat 10 having the configuration shown in FIG. 3.

Further, even when a rug such as a mattress is interposed between the subject 100 and the sensor mat 10, the biological information detection device 1 can detect the respiration rate of the subject 100 with high accuracy.

The present invention is not limited to the embodiments described above, but is also applicable to the embodiments modified in design within the scope of the matters described in the claims. For example, the size and thickness of the first and second polyvinyl chloride plates 31 and 32 and the cushion member 33 are not limited to the examples described above.

The counter 54 shown in FIG. 4 resets the count value every 10 seconds, but the count value may be reset in other cycles. Specifically, the counter 54 may reset the count value every 20 seconds or every 30 seconds. In this case, the biological information detection unit 55 may obtain a respiration rate of 1 minutes by making the count value from the counter 54 3 times or 2 times.

Further, the biological information detecting device 1 can detect not only the breathing rate of the subject 100 but also the heart rate of the subject 100. In this case, the filter characteristic of the filter circuit 52 is set to a characteristic suitable for extracting the heartbeat component of the human body from the signal output from the piezoelectric element 51.

[Comparative measurement 2] We measured the heart rate (pulsation) signal (output signal of filter circuit 52) when the air pad alone was placed under the rug, and the heart rate signal when the sensor mat 10 shown in FIG. 3 was placed under the rug. The measurement conditions were as follows.

The air pad alone and the sensor mat 10 are individually measured.

The air pad alone is the same as the air pad 34 in the sensor mat 10.

The first and second polyvinyl chloride plates 31 and 32 of the sensor mat 10 have a size of 40 cm×40 cm and a thickness of 1 mm.

The thickness of the cushion member 33 (polyurethane material) of the sensor mat 10 is set to 5 mm.

A 2.5 cm thick covering is placed on each of the air pad alone and the sensor mat 10.

A simulated heartbeat signal generator is placed 20 cm from the center of the air pad on the carpet. The pseudo-heartbeat signal generating device is a device that connects an amplifier and a speaker to a function generator to vibrate the speaker at an arbitrary cycle, and generates a pseudo-heartbeat body motion by setting the heartbeat cycle.

The average heart rate of the simulated heart rate signal generator is 84 [cycles/minute].

The weight to be measured is 580 g (1 type). (Because it is difficult to change the weight due to the limitations of the pseudo-heartbeat signal generator)

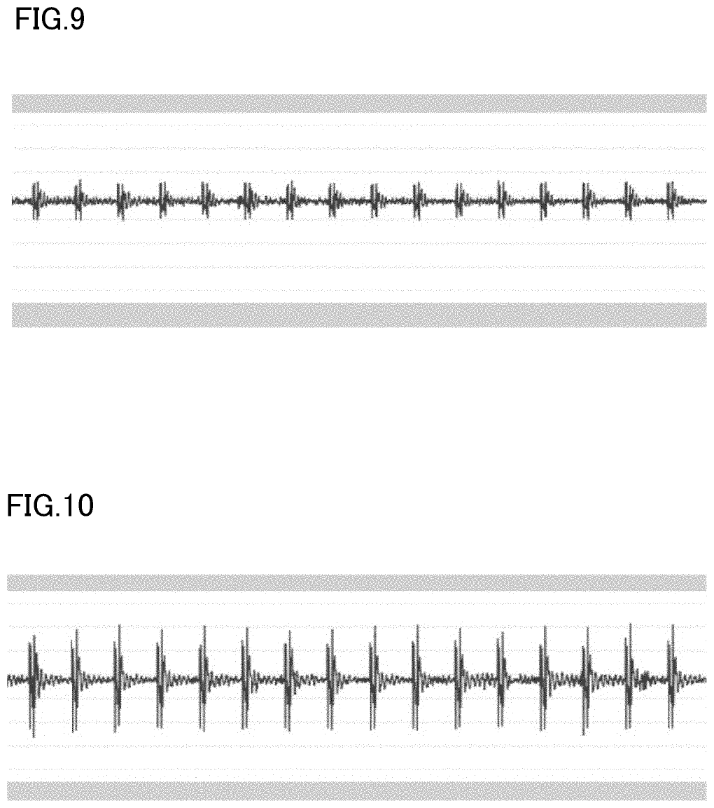

FIG. 9 is a waveform diagram of a heartbeat signal (output signal of the filter circuit 52) when placing the air pad alone under the rug. FIG. 10 is a waveform diagram of a heartbeat signal in the case where the sensor mat 10 shown in FIG. 3 is disposed under a carpet. In FIGS. 9 and 10, the vertical axis direction represents the magnitude of the signal, the horizontal axis direction represents time.

According to FIGS. 9 and 10, the amplitude of the heartbeat signal obtained from the sensor mat 10 is larger than that of the heartbeat signal obtained from the air pad alone. That is, the sensor mat 10 had a higher detection sensitivity for heartbeat than the air pad alone.

As described above, the biological information detection device 1 according to the embodiment of the present invention can detect the heart rate of the subject 100 without using a plate-shaped member having a size covering the entire length of the subject 100 by using the sensor mat 10 having a size equal to or less than half of the total length of the subject 100

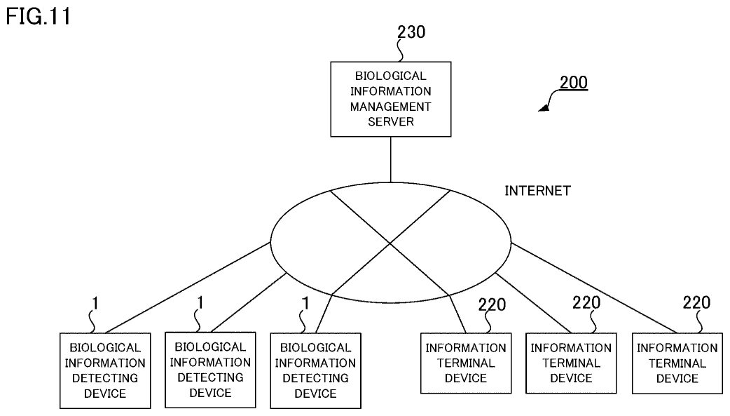

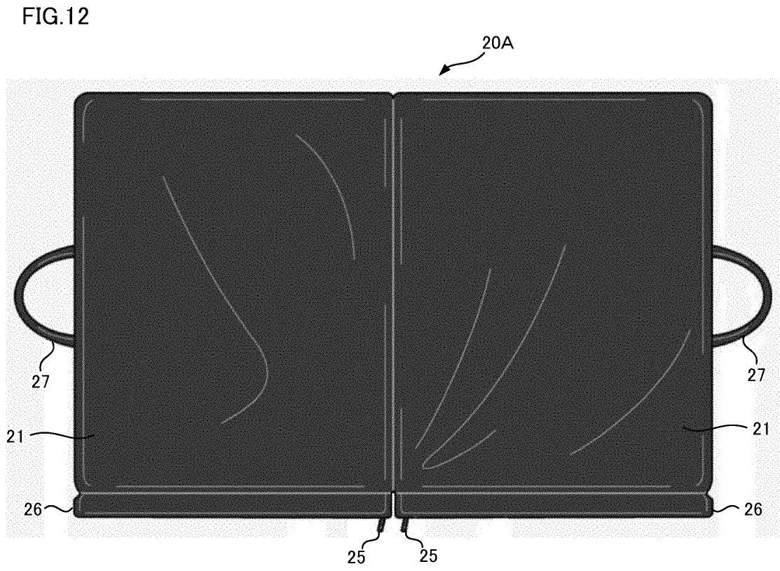

The biological information detecting device 1 can detect the heart rate of the subject 100 with high accuracy even when a mattress or other carpet is interposed between the subject 100 and the sensor mat 10. In addition, the biological information detecting device 1 can simultaneously detect the respiration rate and the heart rate. In this case, the biological information detection device 1 may be provided with each block shown in FIG. 4 for detecting respiration rate and each block shown in FIG. 4 for detecting heart rate.

[A second embodiment] FIG. 11 is the figure showing the composition of the biological information communication system 200 concerning a 2nd embodiment.

The biological information communication system 200 includes a biological information detection device 1 shown in FIG. 1, information terminal devices 220 capable of data communication, and a biological information management server 230 that manages biological information and distributes information on a human body to the information terminal devices 220.

In FIG. 1, three biological information detection apparatuses 1 and three information terminal devices 220 are illustrated, and one biological information management server 230 is illustrated. However, the number of the biological information detection device 1 and the information terminal devices 220 is not particularly limited, and is provided according to the number of subjects who should detect biological information. The biological information management server 230 may be one or a plurality may be provided for load balancing.

The biological information detecting device 1 detects biological information such as a heart rate and a respiration rate from a subject in a sleeping state. Further, the biological information detecting device 1 detects temperature, humidity, brightness of a room, noise, odor, barometric pressure, and the like as environmental information. The biological information detecting device 1 automatically records the detected biological information and environment information in an internal memory, and transmits them to the biological information management server 230 shown in FIG. 1 via the Internet in real time or at a predetermined timing.

In addition, the biological information detecting device 1 stores personal information (age, sex, residential area (positional information), level of care required, etc.) of the subject in advance, and transmits the personal information to the biological information management server 230. The biological information detection apparatus 1 can directly perform data communication with the information terminal devices 220 by wireless communication, infrared communication, or the like without passing through the biological information detection server 30.

The information terminal devices 220 is a terminal that can be connected to an Internet line by wired or wireless. The information terminal devices 220 corresponds to, for example, a so-called smart phone, a tablet terminal, a mobile phone, a small personal computer, or the like. The information terminal devices 220 is owned by a person who wants to confirm the state of the subject, such as a doctor, a nurse, a caregiver, a relative of the subject, or the like.

The information terminal devices 220, the biological information transmitted from the biological information management server 230, the warning information (e.g., warning information indicating that the respiration rate of the subject 100 has fallen below the threshold value for a predetermined time or more continuously, warning information indicating that leaving the floor), and displays other information. Thereby, even when the user is away from the subject, the user can confirm the biological information and other information of the subject, and can grasp the heart rate and the presence or absence of leaving of the subject. Further, the information terminal devices 220 can transmit and receive respective pieces of information of text documents, images, and sounds to and from the biological information detection device 1 via the biological information management server 230.

The information terminal devices 220 can directly communicate with the biological information detection device 1 without passing through the biological information management server 230. In this case, the information terminal devices 220 presents the above-described warning information to the user based on the biological information from the biological information detection device 1.

The biological information management server 230 transmits the biological information transmitted from the biological information detection device 1 to the predetermined information terminal devices 220. Further, the biological information management server 230 can also determine whether the subject's respiratory rate is normal or not, and whether the subject has left the bed or not, based on the biometric information transmitted from the biometric detection device 1.

For example, when the biological information transmitted from the biological information detection device 1 is interrupted for a predetermined time, for example, 20 seconds, the biological information management server 230 determines that the subject has left the bed, and transmits warning information indicating that the subject has left the bed to the information terminal devices 220. In addition, the biological information management server 230 may transmit warning information indicating an abnormality to the information terminal devices 220 when the heart rate transmitted from the biological information detection device 1 is 30 or less or 170 or more consecutively for a predetermined period of time, for example, 2 minutes.

Further, the biological information management server 230 can also compile biological information and personal information from all the subjects connected on the network, and create statistical data (big data) corresponding to the residence area, the season, the level of care required, and the environmental information of the subject. According to this statistical data, it becomes possible to grasp the state of the environmental information of each subject satisfying an arbitrary condition (e.g., male in 60 s, residential area: Hokkaido, level 3 of care required, etc.) if it is stable.

In this case, the biological information management server 230 compares the received information with the statistical data upon receiving the biological information and the environmental information from any biological information detection device 1. Then, when detecting an abnormality (heartbeat, large change in respiration rate, or the like) of the biological information of the subject, the biological information management server 230 transmits warning information for prompting the environment when the biological information is stable to the biological information detection apparatus 1 and the information terminal apparatus 220 based on the statistical data. As a result, the user of the biological information detecting device 1 or the information terminal apparatus 220 can confirm the warning information and adjust the environment (room temperature, humidity, etc.) of the subject to an optimal state.

It is to be noted that the present invention is not limited to the above-mentioned embodiments, and can be applied to those whose design is changed within the scope of the matters described in the claims. The present invention is also applicable to, for example, a smart house with a nursing care service.

The LED lighting equipment in this smart house (indoor) incorporates space/human sensors and infrared sensors. A plurality of LED lighting devices having such a configuration are provided at respective places in the indoor (corridors, baths, toilets, bedrooms, etc.) in a state of being connected to the network, respectively, and detect positional information in the indoor of the patient.

The LED lighting device may communicate with a wristwatch type communication device attached to the patient's arm. The communication device communicates with the LED-lighting device, e.g. by Bluetooth, while being attached to the patient's arms. The LED lighting device detects position information in the indoor of the patient by communicating with the communication device. The position information is transmitted to the biological information management server 230 shown in FIG. 10 via the Internet, and then transmitted to, for example, the information terminal devices 220 of the user.

As a result, the information terminal devices 220 constantly outputs the patient position information based on the patient position information transmitted from the LED lighting device. This allows the user to know not only the patient's health in the bedroom, but also the patient's indoor location and whether the patient has gone outdoors (roaming).

The biological information management server 230 can also perform the following control when the information terminal devices 220 can control the air conditioner, the humidifier, and the dehumidifier (hereinafter referred to as “air conditioner, etc.”) in the smart house. For example, the biological information management server 230 directly controls the air conditioner or the like via the information terminal devices 220 so that the environment is established when the biological information is stable on the basis of the statistical data. As a result, the biological information management server 230 can remotely control the air-conditioning apparatus and the like in the smart house so that the environment of the subject is automatically optimized.

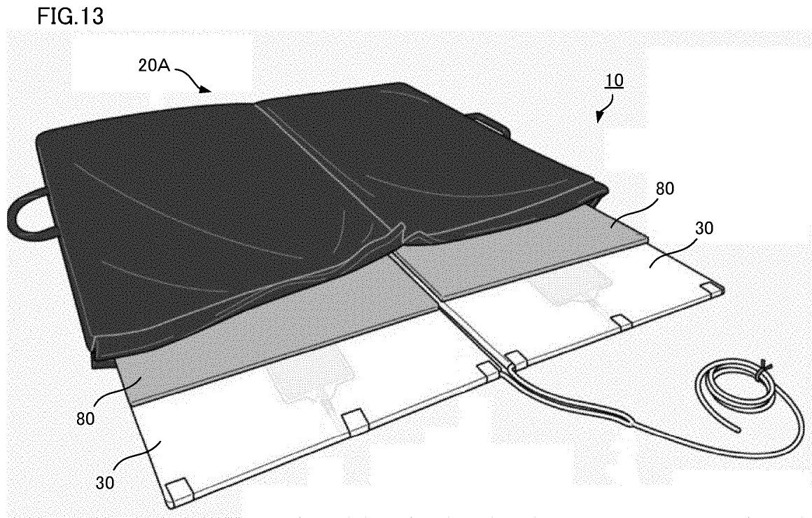

Instead of the sensor mat cover 20 shown in FIG. 2, the following sensor mat cover can also be used. FIG. 12 is a front view showing the sensor mat cover 20A. FIG. 13 is a perspective view showing the sensor mat cover 20A.

The sensor mat cover 20A is a cover configured in a rectangular shape, two bag portions 21 in which the two sensor boards 30 are respectively housed, a fastener 25 for opening and closing the opening of the bag portion 21, a tube protective bag 26 for housing and protecting the tube between the bag portion 21 and the fastener 25, the sensor mat cover 20A and a handle 27 provided on both longitudinal sides respectively. Although not shown, between the bag portion 21 and the tube protective bag 26, the sensor fastener for fixing the sensor board 30 is provided.

Each bag portion 21 is configured to have a shape and size enough to fully accommodate one sensor board 30. Then, each bag portion 21 is disposed so as to be symmetrical with respect to the central axis in the longitudinal direction of the central portion of the sensor mat cover 20. Therefore, the sensor mat cover 20 can be folded into two by the above-mentioned central axis in a state in which the sensor board 30 is accommodated in each bag portion 21.

In each bag portion 21, together with the sensor board 30, a sponge 80 disposed on one surface of the sensor board 30 is accommodated. Then, when the sensor fastener is closed, the position of the sensor board 30 and the sponge 80 within each bag portion 21 is fixed.

The biological information detecting device 1 configured as described above can detect biological information such as the respiration rate with high accuracy not only in adults but also in infants and toddlers of body weight (3 to 10 kg), which is generally difficult to detect.

In addition, in the biological information detecting device 1, if the size of the sensor board 30 (the first and second polyvinyl chloride plates 31 and 32) is enlarged without changing the size of the air pad 34 (see FIG. 3), the detectable range of the biological information of the subject can be enlarged in proportion to the size.

Further, since the sensor board 30 has a sponge 80 inside, it can be used as a mattress alone, and can be used even when it is laid under a mat or a futon.

### REFERENCE SIGNS LIST

1 BIOLOGICAL INFORMATION DETECTOR

10 sensor mat

20, 20A sensor mat cover

30 sensor board

31 first polyvinyl chloride plate

32 second polyvinyl chloride plate

33 cushioning member

34 air pad

50 main unit

55 biological information detector

56 transmitter

200 Biological information and communication system

220 information terminal device

230 biological information management server

1. A mat for detecting biological information, comprising:
an air bag formed in an elongated shape and outputting air according to pressure from a living body;
a plate-shaped cushion member having a thickness substantially the same as that of the air bag, in which the air bag is formed in a central portion, and in which the air bag is fitted; and
a pair of synthetic resin plate-shaped members each having a size substantially the same as that of the plate-shaped cushion member and being formed so as to be bendable as a whole, and provided so as to sandwich the plate-shaped cushion member.

2. The mat for detecting biological information according to claim 1, wherein the air bag has a cushion material having a predetermined thickness therein.

3. A device for detecting biological information, comprising:
a mat for detecting biological information according to claim 1 disposed below a subject; and
a biological information detection unit for detecting periodic biological information of a subject based on air pressure from the mat for detecting biological information.

4. The device for detecting biological information according to claim 3, wherein the mat for detecting biological information outputs air corresponding to a motion transmitted from the living body.

5. The device for detecting biological information according to claim 4, wherein the biological information detection unit detects a respiration rate or a heart rate of the subject as the biological information.

6. The device for detecting biological information according to claim 5, further comprising:
a transmitting unit for transmitting the biological information detected by the biological information detecting unit to an information terminal device.

7. The device for detecting biological information according to claim 6, wherein the information terminal device has an output unit that outputs information based on biological information transmitted from the transmission unit.

8. A system for distributing biological information, comprising:
the device for detecting biological information according to claim 7;
an information terminal device capable of communicating; and
a server device that receives biological information transmitted from the biological information detection device and transmits to the information terminal device at least one of the biological information and information on the state of the detection target person based on the biological information.
